(19) United States
(12) Patent Application Publication
(10) Pub. No.: US 2024/0351962 A1
(43) Pub. Date: Oct. 24, 2024

(54) Catalyst and Process for Conversion of C2-C5 Alkanes to Gasoline Blending Components

(71) Applicant: PURDUE RESEARCH FOUNDATION, West Lafayette, IN (US)

(72) Inventors: Jeffrey Miller, West Lafayette, IN (US); Che-Wei Chang, West Lafayette, IN (US)

(21) Appl. No.: 18/580,682

(22) PCT Filed: May 23, 2022
(86) PCT No.: PCT/US2022/030558

§ 371 Date: Jan. 19, 2024

Related U.S. Application Data
(60) Provisional application No. 63/224,266, filed on Jul. 21, 2021.

(51) Int. Cl. C07C 2/02; B01J 29/44

(52) U.S. Cl. CPC C07C 2/02; B01J 29/44; C07C 2529/44

(57) ABSTRACT

A method for converting lower alkanes to higher liquid products, comprising reacting one or more C₂ to C₁₂ alkanes with a bifunctional catalyst comprising platinum (Pt) or palladium (Pd) and at least one other metal (M) to provide an alloy (Pt-M or Pd-M) containing at least 0.1 wt % of the platinum (Pt) or palladium (Pd), based on a total weight of the catalyst; a silica or alumina support; and an acidic zeolite, at a temperature of about 350° C. to 700° C. to provide a liquid product having a boiling point of 38° C. to 205° C.

### CROSS-REFERENCE TO RELATED APPLICATIONS

This application claims priority to U.S. Provisional Patent Application, having application Ser. No. 63/224,266 that was filed on Jul. 21, 2021, the entirety of which is incorporated by reference herein.

### GOVERNMENT LICENSE RIGHTS

This invention was made with government support under Cooperative Agreement No. EEC-1647722 awarded by the National Science Foundation. The government has certain rights in the invention.

### FIELD OF THE INVENTION

Embodiments provided herein relate to new catalysts and processes for converting light alkanes into higher molecular weight liquid products.

### BACKGROUND OF THE INVENTION

Shale gases production in the U.S has increased the supply of light alkanes, especially methane, ethane and propane. Particularly, ethane and propane could be converted to ethylene and propylene and further transformed into other higher molecular weight hydrocarbons for production of polymers, chemicals and fuels. Since light alkanes are widely distributed across the U.S, many of the production sites require costly transportation over long distances by the pipeline to the East. West and Gulf regions where most processing facilities are located. One attractive option is to develop a process to convert light alkanes (ethane, propane and butanes) into higher molecular weight liquid products (C4+ alkanes, C4+ olefins and aromatics) used as gasoline-blending components, which could be utilized in local markets.

Previously, UOP has developed the Cyclar process utilizing Ga-containing ZSM-5 to convert propane and butane to BTX aromatic chemicals (benzene, toluene and xylene) in a single reactor. The representative Cyclar product distribution is shown in Table 1. Unfortunately, the yield of aromatics has been limited by formation of light gases (mainly methane and ethane), which can't be further transformed into other higher molecular weight liquid products. It remains a challenge to maximize the liquid product yields by suppressing light gases formation. In addition, Cyclar catalysts can't convert ethane at a practical rate. Much effort has further been made in order to develop new catalysts and processes that convert ethane with a much higher rate and liquid product yield.

TABLE 1
product distribution of propane in the Cyclar process

| products | selectivity(%) |
|---|---|
| aliphatic hydrocarbons | ~27 |
| methane, ethane | 22.7 |
| C₂-C₄ olefin (ethylene, propylene, n-butene, isobutene) | 4.4 |
| C₄₊ alkane and olefin | 0.8 |
| aromatics | ~72 |
| benzene | 28.9 |
| toluene | 29.3 |
| C₈ (xylenes, ethylbenzene) | 9.1 |
| C₉⁺ (C₉-C₁₀ alkylbenzene) | 4.7 |

Both the UOP Cyclar process and the process claimed in Shell's patents are designed to produce aromatics chemicals, e.g., benzene, toluene, xylenes, from propane and ethane. In U.S. Pat. No. 8,871,990, No. 9.144.790 and 8,946,107, several bifunctional catalysts with a combination of bimetallic components and acidic zeolites, i.e. platinum/tin (Pt/Sn), platinum/germanium (Pt/Ge), platinum/gallium (Pt/Ga) and platinum/iron (Pt/Fe) containing ZSM-5 have demonstrated better performance for the conversion of ethane to aromatics hydrocarbons. These patents (U.S. Pat. Nos. 8,871,990, 9,144,790 and 8,946,107) indicate that low-Pt/M alloy+ZSM-5 catalysts (M=Sn, Ge, Ga, Fe) provide better initial suppression of methane production. Although the ethane conversion is enhanced, the yield of aromatics is still limited by significant methane production (˜30%).

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are, therefore, not to be considered limiting of its scope, for the invention may admit to other equally effective embodiments. It is emphasized that the figures are not necessarily to scale and certain features and certain views of the figures can be shown exaggerated in scale or in schematic for clarity and/or conciseness.

FIG. 1 depicts the XANES data of Pt/SiO₂ and Pt—Zn/SiO₂.

FIG. 2 depicts the EXAFS data of Pt/SiO₂ and Pt—Zn/SiO₂.

FIG. 3 depicts the methane selectivity as a function of ethane conversion and Pt loading of various bimetallic catalysts, according to one or more embodiments provided herein.

### SUMMARY OF THE INVENTION

Bifunctional catalysts and processes for transforming light hydrocarbons (i.e. ethane/propane) to higher hydrocarbons are provided herein. The bifunctional catalysts include a Pt alloy and acidic zeolites, especially ZSM-5. The catalysts have a high alloy loading and/or high value of alloy to zeolite ratio. The alloy to zeolite ratio is defined as the ratio of the weight loading of the Pt-M or Pd-M on the support (i.e. PtZn/SiO₂ or PtZn/Al₂O₃) to the weight loading of acidic zeolite, preferably ZSM-5.

The bifunctional catalysts provided herein can produce mixtures of paraffins, olefins and aromatics with molecular weights greater than the reactants, e.g., ethane and propane. The bifunctional catalysts provided herein exhibit low methane and ethane selectivity. Much lower methane yields for reaction of ethane and propane to higher molecular weight products with Pt loadings greater than about 0.1% with Pt-Zn+H-ZSM-5 catalysts are also provided.

### DETAILED DESCRIPTION

It is to be understood that the following disclosure describes several exemplary embodiments for implementing different features, structures, or functions of the invention. Exemplary embodiments of components, arrangements, and configurations are described below to simplify the present disclosure; however, these exemplary embodiments are provided merely as examples and are not intended to limit the scope of the invention. Additionally, the present disclosure can repeat reference numerals and/or letters in the various embodiments and across the figures provided herein. This repetition is for the purpose of simplicity and clarity and does not in itself dictate a relationship between the various embodiments and/or configurations. Moreover, the exemplary embodiments presented below can be combined in any combination of ways, i.e., any element from one exemplary embodiment can be used in any other exemplary embodiment, without departing from the scope of the disclosure.

Additionally, certain terms are used throughout the following description and claims to refer to particular components. As one skilled in the art will appreciate, various entities can refer to the same component by different names, and as such, the naming convention for the elements described herein is not intended to limit the scope of the invention, unless otherwise specifically defined herein. Further, the naming convention used herein is not intended to distinguish between components that differ in name but not function.

Furthermore, in the following discussion and in the claims, the terms “including” and “comprising” are used in an open-ended fashion, and thus should be interpreted to mean “including, but not limited to.” The phrase “consisting essentially of” means that the described/claimed composition does not include any other components that will materially alter its properties by any more than 5% of that property, and in any case does not include any other component to a level greater than 3 mass %.

Unless otherwise indicated, all numerical values are “about” or “approximately” the indicated value, meaning the values take into account experimental error, machine tolerances and other variations that would be expected by a person having ordinary skill in the art. It should also be understood that the precise numerical values used in the specification and claims constitute specific embodiments. Efforts have been made to ensure the accuracy of the data in the examples. However, it should be understood that any measured data inherently contains a certain level of error due to the limitation of the technique and/or equipment used for making the measurement.

The term “or” is intended to encompass both exclusive and inclusive cases, i.e., “A or B” is intended to be synonymous with “at least one of A and B,” unless otherwise expressly specified herein.

The indefinite articles “a” and “an” refer to both singular forms (i.e., “one”) and plural referents (i.e., one or more) unless the context clearly dictates otherwise. For example, embodiments using “an olefin” include embodiments where one, two, or more olefins are used, unless specified to the contrary or the context clearly indicates that only one olefin is used.

The terms “alkane” and “paraffin” are used interchangeably and both refer to any saturated molecule containing hydrogen and carbon atoms only, in which all the carbon-carbon bonds are single bonds and are saturated with hydrogen. Such saturated molecules can be linear, branched, and/or cyclic.

The terms “alkene” and “olefin” are used interchangeably and both refer to any unsaturated molecule containing hydrogen and carbon atoms only, in which one or more pairs of carbon atoms are linked by a double bond. Such unsaturated molecules can be linear, branched, or cyclic, and can include one, two, three or more pairs of carbon atoms linked by double bounds (i.e. mono-olefins, di-olefins, tri-olefins, etc).

The term “wt %” means percentage by weight, “vol %” means percentage by volume, “mol %” means percentage by mole, “ppm” means parts per million, and “ppm wt” and “ppmw” are used interchangeably and mean parts per million on a weight basis. All concentrations herein, unless otherwise stated, are expressed on the basis of the total amount of the composition in question.

Each of the appended claims defines a separate invention, which for infringement purposes is recognized as including equivalents to the various elements or limitations specified in the claims. Depending on the context, all references to the “invention” may in some cases refer to certain specific embodiments only. In other cases, it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the art to make and use the inventions, when the information in this disclosure is combined with publicly available information and technology.

It has been surprisingly and unexpectedly discovered that a bifunctional catalyst made from Pt-M or Pd-M alloy with MFI acid sites can produce liquid hydrocarbon products that are highly useful as gasoline blending components. It has also been unexpectedly and surprisingly discovered that such catalyst can provide significantly low methane yields and higher yields of valuable high-octane gasoline blending products from both ethane and propane. The bifunctional catalyst can produce gasoline-blending components instead of aromatic chemicals. Unlike the prior art catalysts that have low Pt loadings with ZSM-5 zeolite to produce aromatics, the bifunctional catalysts provided herein are a high activity catalyst with high Pt or Pd loadings to provide low methane yields (i.e. less than 20% methane, and more preferably less than 5% methane) and high yields of high-octane gasoline blending products (i.e. greater than 80%) from both ethane and propane feed streams.

As explained in more detail below, it has been unexpectedly discovered that alkane monomolecular cracking, which produces methane and other non-reactive alkanes, can be suppressed if there is a sufficient amount of olefins in the product mixture. Olefins are more reactive than alkanes on acid sites, and the presence of olefins in the mixture can hinder alkane reactions on acid sites. The alloy to zeolite ratio, therefore, is significant to control the yield of liquid products (gasoline blending hydrocarbons) by suppressing methane formation. If the alloy to zeolite ratio is too low (lower than 0.02) or the Pt loading is too low (<0.1 wt %), a significant amount of methane can be produced by alkane monomolecular cracking on H-ZSM-5 zeolites and eventually limit the liquid yield. If the alloy to zeolite ratio is too high (higher than 10) or the zeolite loading is too low, the yield of higher molecular weight hydrocarbons will be low because there are not sufficient acid sites to convert the olefins to higher molecular weight hydrocarbons. Balancing the ratio of alloy to zeolite or Pt loading helps control the olefin formation and consumption rate to produce gasoline blending hydrocarbons without making light gases. For example, in the Cyclar process, the rate of propylene formation is low and the rate of olefin consumption to aromatics products on acid sites is high. Thus, in the reacting gas mixture, there are few olefins; as a result, propane reacts on acid sites by a monomolecular cracking pathway to produce ethylene and methane, the latter which is unreactive. Formation of unreactive light gases eventually limits the liquid yield. In this disclosure, we proposed that light gases formation can be suppressed by maintaining olefins in the products to enhance liquid yield. Therefore, ethene and propene will still be present in the product mixture. While ethylene and propylene are not typically blended into gasoline directly, they are still reactive and can be separated from the product mixture and recycled to the reactor to be further converted into gasoline-blending hydrocarbons eventually. The multi-pass liquid yield can exceed 60-70% (reported by Cyclar process); while in the Cyclar process methane remains unreactive even when it is recycled.

As used herein, the term “high-octane gasoline-blending components” refers to a mixture of one or more C₄ to C₁₀ olefins, one or more C₄ to C₁₀ paraffins and one or more C₆ to C₁₀ aromatics, the mixture having a boiling point of 38-205° C. (100-400° F.). The boiling point of the mixture can also range from a low of about 38° C. 45° C., 50° C. or 55° C. to a high of about 180° C., 190° C., 200° C., or 205° C.

By “bifunctional”, it is meant the catalyst has two different catalytic functions, a dehydrogenation function and an olefin conversion function. The bifunctional catalyst can include platinum (Pt), or optionally palladium (Pd), and at least one other metal (M) to provide an alloy (Pt-M or Pd-M) with an acidic zeolite, preferably H-ZSM-5 (MFI). The alloy component provides the dehydrogenation function and the acid sites on the zeolite provide the olefin conversion function, which produces higher molecular weight hydrocarbons by oligomerization, cracking, isomerization and cyclization. Once cyclic paraffins and olefins are formed, these are selectively converted to aromatic products primarily on the dehydrogenation function. The bifunctional catalyst is particularly useful for converting alkanes (paraffins) to alkenes (olefins) and converting the alkenes (olefins) to higher molecular weight products.

The bifunctional catalyst can have a Pt or Pd loading that ranges from 0.1 wt % to 10.0 wt %, based on the total weight of the catalyst (sum of Pt-M or Pd-M alloy on the support, zeolites and the binder). Preferably, the bifunctional catalyst can have a Pt or Pd loading that ranges from 0.1 wt % to 10.0 wt % or preferably about 0.2 wt % to 2 wt %. The Pt or Pd loading can also range from a low of about 0.1 wt %, 1.0 wt % or 2.0 wt % to a high of about 8.0 wt %, 9.0 wt %, or 10 wt %. The M content can range from 0.05 wt % to 15 wt %, based on the total weight of the catalyst. The M content can also range from 2 wt % to 5 wt %. The alloy to zeolite ratio can range from 0.02 to 10. Preferably, the alloy to zeolite ratio can range from 0.1 to 2.

The alloy is not limited to only two metals and can include two or more metals. The metals of the alloy are at least partially present in the metallic phase and/or in a metallic alloy state. At least one other metal (M) can be a transition metal selected from Groups 3-12. In certain embodiments, the metal alloy can be Pt₃Mn, Pt₃Cr and PdZn and other alloys containing Pt or Pd and one other transition metal selected from Groups 5A to 8A and 1B to 5B, for example, V, Mn, Cr, Fe, Co, Ni, Cu, Zn, Ga, Ge, In, Pb, Sn, Sb. or Bi.

The Pt-M or Pd-M alloy can be supported, such as supported on a silica (SiO₂) or alumina (Al₂O₃) supports, and then physically mixed with at least one acidic zeolite. The bifunctional catalyst extrudate can be shaped into cylinders from about 1/16 to ⅛″ with a length of about 0.25 to 0.5″. The ZSM-5 plus support powders can be ground in different ratios and pelletized or extrududed prior addition of the PtM or Pd M alloy. Silica (SiO₂) and alumina (Al₂O₃) supports are preferred, but other additional supports may be used, such as silica-alumina, magnesium aluminate (MgAl₂O₄), clays, zeolites and carbon.

The alloy support material can have a surface area in the range of from about 10 m²/g to about 700 m²/g, a pore volume in the range of from about 0.1 cc/g to about 4.0 cc/g and an average particle size of the support powders in the range of from about 5 μm to about 500 μm. More preferably, the support material can have a surface area in the range of from about 50 m²/g to about 500 m²/g, pore volume of from about 0.2 cc/g to about 3.5 cc/g and average particle size of from about 10 μm to about 200 μm. The surface area can range from a low of about 50 m²/g, 150 m²/g, or 300 m²/g to a high of about 500 m²/g, 700 m²/g, or 900 m²/g. The surface area also can range from a low of about 200 m²/g, 300 m²/g, or 400 m²/g to a high of about 600 m²/g, 800 m²/g, or 1.000 m²/g. The average pore size of the support material can range of from about 10 Å to 1000 Å, about 50 Å to about 500 Å, about 75 Å to about 350 Å, about 50 Å to about 300 Å, or about 75 Å to about 120 Å.

The present disclosure is not limited by the method of catalyst preparation. All suitable methods should be considered to be within the scope herein. Conventional methods include co-precipitation from an aqueous, an organic, or a combination solution-dispersion, impregnation, dry mixing, wet mixing or the like, alone or in various combinations. In general, any method can be used which provides compositions of matter containing the prescribed components in effective amounts. According to an embodiment the substrate is charged with active metal via an incipient wetness impregnation. Other impregnation techniques such as by soaking, pore volume impregnation, or percolation can optionally be used. Alternate methods such as ion exchange, wash coat, precipitation, and gel formation can also be used.

When slurries, precipitates or the like are prepared, they will generally be dried, usually at a temperature sufficient to volatilize the water or other carrier, such as from 100° C. to 250° C., with or without vacuum, irrespective of how the components are combined and irrespective of the source of the components, the dried composition can be calcined in the presence of a free oxygen-containing gas, usually at temperatures between about 200° C. and about 600° C. for from 1 to 24 hours. The calcination can be in an oxygen-containing atmosphere, or alternately in a reducing or inert atmosphere.

The acid sites in the bifunctional catalyst can be provided by one or more acidic zeolites, preferably ZSM-5. The three-dimensional framework structure of ZSM-5 zeolites has straight channels (5.4×5.6 Å) interconnected by sinusoidal channels (5.1×5.5 Å) with silica to alumina ratios (Si/Al) of 12 or more. Preferably, the Si/Al ratio of ZSM-5 ranges from 12 to 160.

Acid sites can catalyze a variety of olefin reactions, including oligomerization, cracking, isomerization and cyclization; however, acid sites can also catalyze undesired alkane monomolecular cracking, which leads to light gas formation (particularly methane). Preferentially, the acid sites are used to convert olefins, which are formed by alkane dehydrogenation on the dehydrogenation sites, to higher molecular weight hydrocarbons; while, methane formation by alkane monomolecular cracking on acid sites is suppressed. The high Pt loading or high alloy to zeolite ratio of the bifunctional catalyst provided herein can maintain an olefin concentration in the product mixture so that the acid sites convert olefins to liquid products but not convert alkanes to methane.

Binder material, extrusion aids or other additives can be added to the catalyst composition or the final catalyst composition. The binder is added to the zeolite component to provide physical strength to the catalyst particle. For example, the composite catalyst can include a binder of silica, alumina, silica-alumina, magnesium-aluminate, clays or other metal oxides as a binder. Upon calcination these elements can be altered, such as through high temperature calcination to increase the crush strength of the final catalyst structure. The amount of binder can be from about 5 to 95%, preferably from 20 to 80%. Often the binder is a high surface area, refractory oxide, which can also be used to deposit the Pt-M or Pd-M alloy. Most often, the Pt-M or Pd-M alloys are added after calcination of the extruded binder zeolite particles.

The prepared catalyst can be ground, pressed, sieved, shaped and/or otherwise processed into a form suitable for loading into a reactor. The reactor can be any type known in the art, such as a fixed bed, fluidized bed, or swing bed reactor. Optionally an inert material, such as quartz chips, can be used to support the catalyst bed and to locate the catalyst within the bed. Depending on the catalyst, a pretreatment of the catalyst may, or may not, be necessary. For the pretreatment, the reactor can be heated to elevated temperatures, such as 200° C. to 900° C. with an air flow, such as 100 mL/min, and held at these conditions for a length of time, such as 15 min to 3 hours. Then, the reactor can be brought to the operating temperature of the reactor, for example 150° C. to 900° C., or optionally down to atmospheric or other desired temperature. The reactor can be kept under an inert purge, such as under a nitrogen or helium purge. The catalyst can be reduced in H₂ at temperature from 300 to 700° C. prior to reaction.

The bifunctional catalyst provided herein can be used to convert an alkane feedstock to provide one or more gasoline blending hydrocarbons (olefins, paraffins and aromatics). In one embodiment, the alkane feedstock can be introduced into a reaction chamber and contacted with the bifunctional catalyst at reaction conditions sufficient to provide a product containing a mixture of olefins, paraffins and aromatics. As mentioned above, the bifunctional catalyst can be regenerated in-situ, when necessary. Optionally, any paraffins and olefins in the product can be recycled to the feedstock to alter the process distribution and molecular weight distribution of the product.

The alkane feedstock can contain one or more alkanes having less than 10 carbon atoms. The feedstock can consist essentially of C₂-C6 alkanes. An embodiment of the invention provides for the use of ethane or propane or butane or a mixture of these gases as the starting material. Embodiments of the invention are particularly suitable for the production of ethene or propene or butenes or a mixture of these olefins. The alkane feedstock can be obtained from the side product of various hydrocarbon processing plants, for instance, the offgas of an FCC cracker or other refinery processes, refinery fuel gas, or shale gas hydrocarbons. One source of alkane feedstock is from natural gas liquids (GL's) that can be extracted by gas processing plants, often a cryogenic process that extract the NGL's from a gas stream, such as a gas stream produced from a shale formation. One source of alkane feedstock is liquid petroleum gas (LPG), which consists mainly of the propane and butane fraction and can be recovered from gas and oil fields and petroleum refining operations. The alkanes can be co-fed with a stream of H₂ and/or inert gas. Steam can also be co-fed if desired as a diluent or as a heat transfer agent, either way, steam is inert. Since the catalyst can withstand steam at the temperatures used for this process, steam can be used as a co-feed to increase conversion while reducing coke formation. The H₂:alkane ratio or inert:alkane ratio can range from about 0 to 5, optionally 0 to 2.0.

In an illustrative embodiment the alkane feed can contain primarily ethane. In an illustrative embodiment the alkane feed can contain primarily propane. In an illustrative embodiment the alkane feed can contain primarily butane. In an illustrative embodiment the alkane feed can contain primarily ethane and propane. In an illustrative embodiment the alkane feed can contain primarily propane and butane. In an illustrative embodiment the alkane feed can contain primarily butane and pentane. In an illustrative embodiment the alkane feed can contain primarily C3-C6 alkanes. In an illustrative embodiment the alkane feed can contain primarily C2-C6 alkanes.

The catalysts described herein may be used in any suitable reactor. The process could utilize a series of fixed bed reactors, where each reactor could be independently regenerated, a moving bed reactor where the catalysts moves through the reactor and is regenerated in a separate section of the plant, or a fluidized bed reactor, where the catalyst is circulated through the reactor and regenerated in a separate vessel. The operation conditions (temperature, pressure and space velocity) are also significant for controlling product selectivity. For example, the alloy to zeolite site ratio can be 0.02-10 or Pt loading (0.1-10 wt %) at 550° C. and 1 atm on PtZn+ZSM-5 catalysts for propane conversion to higher molecular weight hydrocarbons without making much light gases (i.e. less than 3 wt %). At higher temperature (>550° C.) and 1 atm, the alloy to zeolite site ratio or Pt loading can vary based on the olefin formation and consumption rates on the alloy and zeolites.

Space velocity is another consideration to achieve high liquid yield. If space velocity is too high, olefins conversion into higher molecular weight hydrocarbons will be too low. If space velocity is too low, olefins can be consumed too rapidly and the absence of olefins will lead to methane formation.

The reaction conditions can also be considered along with the catalyst composition (alloy to zeolite ratio or Pt loading) to achieve high liquid yield. The reaction can take place at a temperature of from 350° C. to 1000° C., optionally from 400° C. to 800° C., optionally from 450° C. to 750° C. For example, the reaction may take place at up to 400° C., 450° C., 500° C., 550° C., 600° C., 650° C., 700° C., 750° C., 800° C., 850° C., 900° C., 950° C., or 1000° C. The pressure can be in the range of from 3 psig to 600 psig, optionally from 3 psig to 300 psig, optionally from 3 psig to 150 psig. The weight hourly space velocity can be from 0.3 to 1000 hr⁻¹, optionally from 0.3 to 10 hr⁻¹, and optionally from 0.5 to 3 hr⁻¹.

The reactions can be performed adiabatically or non-adiabatically or approximately isothermally. If the reactions are performed in an adiabatically operated catalyst bed, an endothermic reaction will cause the temperature to decrease over the length of the catalyst bed. The reaction rate in the catalyst bed is thus limited so that several catalyst beds are typically required to achieve the desired high reaction rates and re-heating is necessary downstream of each catalyst bed. In order to achieve reasonable reaction rates, several catalyst beds are normally arranged in series and the reaction system is re-heated downstream of each catalyst bed.

If the reactions are performed in a non-adiabatically operated catalyst bed, the catalyst bed can be heated in order to maintain a high temperature. Because the temperature in the reaction system is kept constant, the reaction rates may be kept appropriately high. Because of the location of the point of thermodynamic equilibrium, however, the disadvantage is that these high reaction rates can only be achieved at high temperatures, as a result of which the selectivity of olefin formation may be reduced. Hence, consecutive reactions will increasingly take place, so that undesired products may form, such as CH₄. C2H4, C2H6 and coke. The by-products thus formed, especially finely dispersed coke, can precipitate in the course of the reaction on the catalyst, thus causing its state to change continually. The catalyst becomes coated with an undesired substance and is thus less accessible for the reactants. This means that the catalyst becomes deactivated. The activity of the catalyst for alkane dehydrogenation and the selectivity for alkene formation may in turn deteriorate. This would result in deterioration of the efficiency of the process as a whole. Because of operational requirements, such a deactivation can only be tolerated up to certain limit, because an economically viable operation of the plant could no longer be guaranteed. In order to counter-act this negative influence on the process, the catalyst will have to be regenerated after a certain reaction period in order to recover its activity.

Depending on its characteristics, the bifunctional catalyst can be regenerated by bringing it in contact with an oxygen-bearing gas under conditions defined for the regeneration of the catalyst. The conditions for such a regeneration may differ from those required for the reactions. An oxygen-bearing gas diluted with steam may also be fed through the catalyst. As a result of this procedure, the by-products on the catalyst are reduced, with the result that the catalyst can regain its activity. If an oxygen-bearing gas diluted with steam is used for catalyst regeneration, the carbon-bearing deposit reacts to form carbon dioxide as the main product. The carbon-bearing deposit is converted to gaseous products by this reaction and is removed from the system. In an embodiment, the bifunctional catalyst can undergo in-situ regeneration, which can lower operating costs by decreasing the amount of time the reactor must be offline. The regeneration can be done at the reaction temperature by burning of carbon with oxygen concentrations between 0.1 and 20%, optionally from 0.3-10%, and optionally from 0.5 to 3%. Alternatively, the catalyst can be regenerated with hydrogen at the reaction temperature. In an embodiment the bimetallic catalyst can undergo ex-situ regeneration.

As the conditions for the reaction process differ from the catalyst regeneration process, the reaction process can be interrupted after a certain period of operation and substituted by the catalyst regeneration process. Thereafter, the reactor bed can be purged and again made available for the reaction. Both these processes, i.e. the reaction and catalyst regeneration, are thus performed periodically. In order to render the overall process economically efficient, this can take place in two or a plurality of catalyst beds, in which the reaction and regeneration processes are alternately implemented. In order to ensure optimum catalyst regeneration, regeneration process should be instrumented and monitored.

The liquid product can contain a mixture of one or more olefins, one or more paraffins and/or one or more aromatics. For example, the product mixture can include about 5 wt % to about 80 wt % of one or more olefins; about 5 wt % to about 80 wt % of one or more paraffins; and/or about 10 wt % to about 90 wt % of one or more aromatics. The product mixture can also include about 5 wt % to about 30 wt % ethylene; about 10 wt % to about 50 wt % propylene; 0.0 wt % to about 5 wt % butane and isobutane; 0 wt % to about 10 wt % butenes; 0.0 wt % to about 5 wt % C5+; about 5 wt % to about 40 wt % benzene; about 1 wt % to about 30 wt % toluene; 0 wt % to about 15 wt % C8+ aromatics. The product mixture can also contain less than 10 wt % methane, less than 30 wt % ethane; and less than 15 wt % propane. Preferably, the product mixture contains less than 5 wt % methane.

The product mixture should contain olefins above 15 wt % to suppress the undesired monomolecular cracking. For example, the product mixture from ethane can contain minimal 20 wt % to maximal 60 wt % ethylene; minimal 0 wt % to maximal 20 wt % propylene; minimal 0 wt % to maximal 10 wt % butenes. For example, the product mixture from propane can contain minimal 5 wt % to maximal 30 wt % ethylene; minimal 5 wt % to maximal 40 wt % propylene; minimal 0 wt % to maximal 20 wt % butenes. The olefins remaining in the product mixture can be recycled to further improve the yield of gasoline-blending hydrocarbons.

The reaction products can be processed and separated by cooling or other standard recovery or separation techniques.

### EXAMPLES

Embodiments discussed and described herein can be further described with the following examples. Although the following examples are directed to specific embodiments, they are not to be viewed as limiting in any specific respect.

### Example 1: Pt—Zn on SiO₂ Catalyst Preparation

The silica (Davisil 646) was purchased from Sigma-Aldrich. The pore volume was determined by adding water dropwise to a known amount of SiO₂ until it was saturated and was 1.16 mL/g, 10 g SiO₂ was weighed. Separately, 1.36 g Zn(NO₃)₂·6H₂O was dissolved in 100 mL deionized water to obtain 3% Zn/SiO₂. Subsequently, ammonium hydroxide (NH₄OH) is added to Zn(NO₃)₂ first precipitating a white Zn oxide/hydroxide solid. Additional NH₄OH was added producing a clear solution with a pH measured by pH paper about pH of 11-12. The SiO₂ was added to the Zn solution and stirred for 10 minutes. The sample was vacuum filtered and washed three times with 100 mL deionized water. The wet powder was dried overnight at 125° C. and calcined at 300° C. for 3 hr (10° C./min).

Pt was added to the Zn/SiO₂ by incipient wetness impregnation method (IWI) to give 2% Pt in the final catalyst. Next, 0.4 g Pt(NH₃)₄(NO₂)₂ was dissolved in about 9.1 mL of deionized water. Two (2) mL ammonium hydroxide was added to the Pt solution and stirred until all crystals dissolved. The pH of the Pt solution was about 11-12. The solution was added dropwise to the Zn/SiO₂ support. The catalyst was dried overnight at 125° C., calcined at 200° C. for 3 h (5° C./min ramp) and reduced at 225° C. in 5% H₂/N₂ at 100 cm³/min for 30 min. The Pt—Zn catalysts were evaluated at different space velocities for propane dehydrogenation at different conversions at 550° C. to ensure >97% propylene selectivity at conversions of about 30% before being mixed with acidic zeolites for preparation of the bifunctional catalyst. PtZn alloy catalysts on alumina supports with identical structure and catalytic performance were prepared by the same synthesis procedure.

### Pt—Zn Alloy Structure Characterization

In situ X-ray absorption spectroscopy (XAS) experiments were performed at the 10-BM-B beamline at the Advanced Photon Source synchrotron at Argonne National Laboratory at the Pt L3 edge (11.564 keV) to determine the structure information of the bimetallic alloy prepared above. Samples were placed in a quartz tube sealed with leak-tight end caps, reduced in 5% H₂/N₂ at 550° C. and cooled in helium. Each measurement was accompanied by a Pt foil scan which was obtained through a third ion chamber and used for calibration. The X-ray absorption near edge structure (XANES) and extended X-ray absorption fine structure (EXAFS) data were obtained in the XAS spectrum. XANES provided information about the oxidation state and coordination environment of specific element according to edge energy, which was determined by the first inflection point. The EXAFS spectra, interpreted by scattering from neighbor atoms, was used to find the coordination number (CN) and bond distance. The data of XANES and EXAFS were interpreted using WinXAS 3.1 software to understand the oxidation state, coordination number and bond distance.

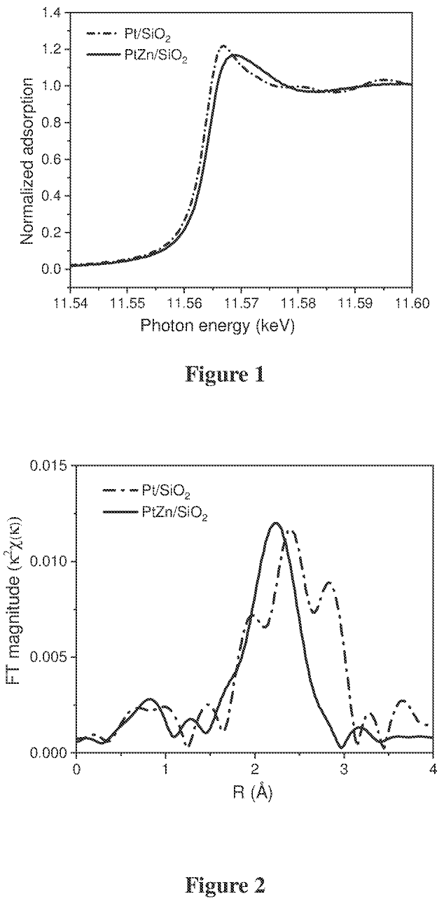

FIG. 1 depicts the XANES data of Pt/SiO₂ and Pt—Zn/SiO₂. Comparing the XANES of the Pt—Zn bimetallic alloy with a comparative monometallic Pt catalyst (Comp. Ex.1) at Pt L3 edge (FIG. 1), the edge energy of Pt—Zn/SiO₂ bimetallic alloy was 0.9 eV higher energy than Pt and has a different shape consistent with formation of Pt₁Zn₁ alloy (structure type of Au₁Cu₁).

FIG. 2 depicts the EXAFS data of Pt/SiO₂ and Pt—Zn/SiO₂. In the EXAFS spectrum, the three peaks of Pt catalyst in the magnitude of the k2-weighted Fourier transform (FT) were characteristic peaks of metallic Pt (FIG. 2). However, the Pt—Zn alloy had only one peak, which is distinctly different from metallic Pt. The EXAFS fit of Pt—Zn/SiO₂ was consistent with the Pt₁Zn₁ phase with a tetragonal Au₁Cu₁ structure, suggesting a Pt—Zn intermetallic full alloy was formed.

The k²-weighted EXAFS at the Pt edge of Pt/SiO₂ and Pt—Zn/SiO₂ were fitted to acquire the average coordination number and bond distance between Pt and the nearest neighbor atoms (Table 2). Pt—Pt with an average bond distance of 2.74 Å and coordination number of 9.3 was confirmed on Pt/SiO₂ catalyst. The average Pt—Zn bond distance of 2.57 Å and coordination number of 5.5 were obtained on Pt—Zn/SiO₂. The coordination number (CN) of Pt—Zn/SiO₂ was smaller than that in bulk crystal Pt₁Zn₁ (CN=8), implying small particles are formed.

TABLE 2
Coordination number and bond distance from in
situ EXAFS simulation of Pt/SiO₂ and Pt—Zn/SiO₂

| | Sample | Scattering | Coordination | Bond | |
|---|---|---|---|---|---|
| | Name | Pair | Number | Distance (Å) | Δσ2 |
| Comp. | Pt | Pt—Pt | 9.3 | 2.74 | 0.001 |
| Ex. 1 | | | | | |
| Ex. 1 | Pt—Zn | Pt—Zn | 5.9 | 2.57 | 0.007 |

### Example 2: Propane Conversion over the Pt-Zn+H-ZSM-5 Catalysts (Catalysts A-D)

Four bifunctional catalysts (Catalysts A-D), according to one or more embodiments of the invention, were prepared according to the procedure described above in Example 1 (Pt—Zn/SiO₂ bimetallic alloy catalysts). Catalysts A-D were each physically mixed with pelleted ZSM-5 at the different ratios shown in Table 3 below. The pelleted ZSM-5 was prepared by grinding a ZSM-5 extrudate into fine powder and pelletized prior to mixing in different ratios of the catalyst. The ZSM-5 extrudate (CBV 5524G CY (1.6) was obtained from Zeolyst International, Inc. and was 80 wt % H-ZSM-5 zeolite with SiO₂/Al₂O₃=50 and 20 wt % Al₂O₃ binder.

### Catalytic Performance Evaluation

Catalytic performance of the catalysts was evaluated by performing the ethane/propane conversion in a 10.5 mm ID quartz tube fixed bed reactor equipped with a mass flow rate controller. The catalysts were supported on quartz wool with an internal thermocouple monitoring the temperature of the catalyst bed. The products were analyzed with a Hewlett Packard 6890 gas chromatograph with Agilent J&W HP-1 GC Column using a flame ionization detector (FID). Initially, the catalyst was purged with ultra-high purity nitrogen (N₂) to remove any adsorbed moisture and reduced in hydrogen (H₂) at 550° C. for 1 hour before the reaction. Catalysts A to D were then tested at 550° C. and atmospheric pressure using 5% propane/N₂.

The catalyst composition, initial propane conversion and product distribution are reported in Table 3 below. Table 3 also reports the product distributions of catalysts A to D, which were olefins and benzene, toluene, and xylene (BTX) aromatics with little methane.

TABLE 3
Product distribution of 5% propane conversion at 550° C.

| | Comp. Ex. 1 | Comp. Ex. 2 | | | | |
|---|---|---|---|---|---|---|
| Catalyst | ZSM-5 | Pt—Zn/SiO₂ | Ex. A | Ex. B | Ex. C | Ex. D |
| catalyst weight (g) | 0.5 | 0.05 | 0.35 | 0.7 | 0.1 | 0.2 |
| Pt loading (wt %) | 0 | 2.0 | 0.29 | | 1.0 | |
| Zn loading (wt %) | 0 | 3.0 | 0.43 | | 1.5 | |
| ZSM-5 loading (wt %) | 80.0 | 0 | 68.6 | | 40.0 | |
| Binder (wt %) | 20 (alumina) | 95 (silica) | 30.7 (silica) | | 57.5 (silica) | |
| Propane conversion (%) | 44.0 | 36.0 | 46.4 | 74.7 | 47.8 | 72.4 |
| Volumetric flow rate | 66 | 66 | 66 | 66 | 33 | 33 |
| (cm³/min) | | | | | | |
| Space velocity (mol/g/s) | 4.5 × 10⁻⁶ | 4.5 × 10⁻⁵ | 6.4 × 10⁻⁶ | 3.3 × 10⁻⁶ | 1.1 × 10⁻⁵ | 5.6 × 10⁻⁶ |
| | Carbon selectivity (%) | | | | | |
| Methane | 26.0 | 0.2 | 4.4 | 4.4 | 1.9 | 2.5 |
| Ethane | 3.3 | 0 | 4.4 | 14.4 | 2.4 | 11.3 |
| Ethylene | 40.4 | 0.3 | 14.2 | 7.0 | 8.1 | 5.4 |
| Propylene | 16.8 | 99.5 | 37.9 | 19.8 | 60.2 | 39.9 |
| Butane, Isobutane | 0.6 | 0 | 0.4 | 0.4 | 0.4 | 0.6 |
| Butenes | 2.5 | 0 | 3.7 | 2.0 | 4.2 | 3.3 |
| C₅^{₊} | 0.6 | 0 | 0.8 | 0.3 | 1.0 | 0.8 |
| Benzene | 4.9 | 0 | 13.5 | 22.0 | 10.8 | 16.0 |
| Toluene | 3.7 | 0 | 15.9 | 23.2 | 8.4 | 15.8 |
| C₈₊ aromatics | 1.3 | 0 | 4.8 | 6.4 | 2.6 | 4.4 |

As shown above, it was surprisingly and unexpectedly discovered that a higher loading of Pt—Zn alloy (high alloy to zeolite ratio) was able to suppress methane formation and increase the yield of higher molecular weight products (C₄⁺ hydrocarbons and BTX aromatics). As shown in Table 3, the bifunctional catalysts A-D surprisingly and unexpectedly demonstrated much higher selectivity to gasoline range hydrocarbons than either the single component ZSM-5 or PtZn alloy catalyst. The single component ZSM-5 (comparative example 1) had a 44% propane conversion and methane selectivity of about 26%. The single component Pt—Zn alloy catalyst (comparative example 2) had a 36% propane conversion, a propylene selectivity of near 99% and less than 1% methane selectivity, without forming higher molecular weight hydrocarbons. Its selectivity to high octane aromatics was 9.9%. At a similar conversion to that of ZSM-5, for example, of 4648%, the bifunctional catalysts A and C had a methane selectivity of about 4% and 2% with BTX selectivities of 34 and 22%, respectively. At higher conversions, the bifunctional catalysts B and D had a methane selectivity of about 4% and about 2.5%, respectively, and the BTX selectivities increased to 52% and 36%, respectively. Catalysts A and B (0.3 wt % Pt) demonstrated about 4.4% methane selectivity while catalysts C and D (1 wt % Pt) demonstrated 1.9% and 2.5%, respectively, demonstrating that high Pt-M alloy levels in the catalyst give low methane selectivity.

### Example 3: Ethane Conversion Over the Pt-Zn+H-ZSM-5 Catalysts (Catalysts E-G)

Three additional bifunctional catalysts (Catalysts E to G) were prepared according to the catalyst preparation procedure of Example 1 and tested at 600° C., atmospheric pressure for 100% ethane conversion. Catalysts E and F had a high Pt loading, while comparative example catalyst 3 has low Pt loading (0.1 wt %). Table 4 presents the catalyst compositions, initial ethane conversion and product distribution.

TABLE 4
Product distribution of 100% ethane conversion at 600° C.

| | Ex. E | Ex. F | Comp. Ex. 3 |
|---|---|---|---|
| Catalyst | | | |
| catalyst weight (g) | 0.12 | 0.3 | 0.21 |
| Pt loading (wt %) | 9.0 | 1.0 | 0.1 |
| Zn loading (wt %) | 13.5 | 1.5 | 0.15 |
| ZSM-5 loading (wt %) | 13.4 | 53.4 | 76.2 |
| Silica Binder (wt %) | 64.1 | 44.1 | 23.5 |
| Ethane conversion (%) | 28.5 | 30.1 | 22.4 |
| Volumetric flow rate (cm³/min) | 33 | | |
| Space velocity (mol/g/s) | 1.9 × 10⁻⁴ | 7.4 × 10⁻⁵ | 1.1 × 10⁻⁴ |
| Carbon selectivity (%) | | | |
| Methane | 1.8 | 3.1 | 3.4 |
| Ethylene | 79.4 | 54.7 | 53.2 |
| Propane | 2.2 | 3.8 | 3.1 |
| Propylene | 4.1 | 7.5 | 13.6 |
| Butane, Isobutane | 0.7 | 0.7 | 1.1 |
| Butenes | 2.9 | 1.8 | 2.6 |
| C₅⁺ | 0.8 | 0.7 | 1.2 |
| Benzene | 4.6 | 13.6 | 10.0 |
| Toluene | 2.7 | 10.9 | 8.9 |
| C₈+ aromatics | 0.9 | 3.2 | 2.8 |

As shown in Table 4 above, methane selectivity was surprisingly low with a high loading of the Pt—Zn alloy. Catalyst E with 9% Pt loading produced less than 2% methane. Ethane conversion of Catalysts E and F was higher than for comparative catalyst 3 with 0.1 wt % Pt loading. The BTX selectivity of catalyst F was higher than that of comparative catalyst 3. By comparing catalysts E. F and G, it was also found that methane selectivity decreased from 3.4% (comp. catalyst 3) to 1.8% (catalyst E) as the Pt loading increased from 0.1 wt % (comp. catalyst 3) to 9 wt % (catalyst E), which further illustrated that higher Pt loading surprisingly and unexpectedly minimizes methane formation, as discovered above with Catalysts A-D focused on propane conversion as reported in Table 3.

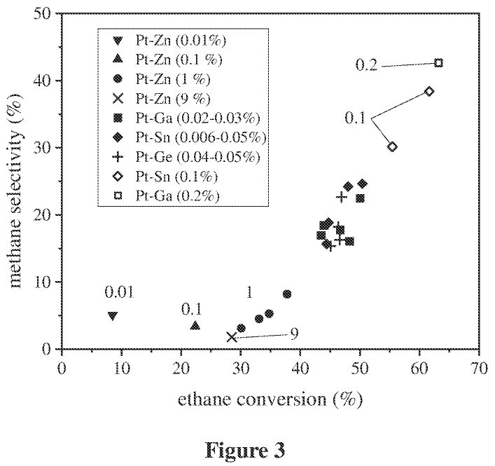

FIG. 3 shows the methane selectivity as a function of ethane conversion over the Pt-Zn+H-ZSM-5 catalysts (E,F, comp. Ex 3) in comparison with low Pt/M/ZSM-5 catalysts (M=Sn, Ge, Ga), which contain less than 0.05 wt % Pt based on the total weight of the catalyst (red solid square). As shown, catalysts E and F have better initial suppression of methane production. Catalysts E and F with higher Pt loading (e.g. >0.05 wt %) unexpectedly showed that methane formation can be suppressed using higher loading Pt alloy (>0.1 wt %).

Not wishing to be bound by theory, it has been surprisingly and unexpectedly discovered that the difference in selectivity to light gas formation compared to the prior art is the presence of olefins in the products. In the absence of olefins in the final product, which occurs when aromatic products like BTX are produced, intermediate alkanes are cracked by the ZSM-5 catalyst. When the product distribution contains olefins, ZSM-5 catalyst's high reactivity greatly suppresses this non-selective acid cracking of alkanes giving lower light gas and higher liquid yields. In addition to the higher Pt alloy loading, which gives higher rates of olefin formation, it is also important to control the number of acid sites in the zeolite fraction as well as the operating conditions. The amount of acid sites in the bifunctional catalyst can be controlled by adjusting the weight loading of ZSM-5, for example. The operating conditions can be controlled by adjusting reaction temperature, pressure and space velocity. Controlling the amount and activity of zeolite fraction can control the olefin consumption rate so that olefins can remain in the product mixture. Adjusting alloy to zeolite ratio can also control the olefin rate (generation and consumption) on the Pt alloy and acid zeolites. Low methane selectivity and higher liquid yields were obtained when the product contained olefins as well as paraffins and aromatics exiting the reactor.

### Low Temperature Propane Conversion Over the Pt-Zn+H-ZSM-5 Catalysts

Catalyst D was further evaluated at 350° C. and 550° C. using 100% propane at atmosphere pressure (Table 5). Catalyst D was able to convert pure propane into high yield of higher molecular weight hydrocarbons with little methane formation (<5%) at 550° C. The products at 550° C. included ethane, olefins and BTX aromatics where ethane and olefins were recycled back to the reactors to further increases the final yield. Catalyst D was also able to convert pure propane gas to 93% selectivity to higher molecular weight hydrocarbons at about 9% propane conversion to C4, C5⁺ and aromatic hydrocarbons at low temperature (350° C.).

TABLE 5
Product distribution of 100% propane conversion
at 350° C. and 550° C.

| | D |
|---|---|
| Catalyst | |
| catalyst weight (g) | 0.2 |
| Pt loading (wt %) | 1.0 |
| ZSM-5 loading (wt %) | 40.0 |

| | | |
|---|---|---|
| Temperature (° C.) | 350 | 550 |
| Propane conversion (%) | 8.7 | 66.8 |
| Volumetric flow rate (cm³/min) | 33 | 66 |
| Space velocity (mol/g/s) | 1.1 × 10⁻⁴ | 2.2 × 10⁻⁴ |
| Carbon selectivity (%) | | |
| Methane | 0.6 | 4.6 |
| Ethane | 6.4 | 25.7 |
| Ethylene | 0.5 | 1.7 |
| Propylene | 11.2 | 15.5 |
| Butane, Isobutane | 47.1 | 5.3 |
| Butenes | 8.3 | 3.8 |
| C₅⁺ | 18.0 | 0.9 |
| Benzene | 0.7 | 14.6 |
| Toluene | 3.1 | 19.4 |
| C₈₊ aromatics | 4.1 | 8.5 |

At 350° C. the propane conversion was unexpectedly higher than the dehydrogenation equilibrium to propylene. This likely occurred because the initially formed propylene was converted to higher molecular weight products allowing for increased propane conversion. In addition, the product distribution at 350° C. was distinctly different than the product distribution at 550° C. The product distribution at 350° C. exhibited paraffins (ethane, butanes and pentanes) and BTX aromatics with the BTX aromatics selectivity even higher than that of 550° C. at the same conversion.

Other specific embodiments provided herein further include the following numbered paragraphs:
1. A method for converting lower alkanes to higher liquid products, comprising: reacting one or more C₂ to C₁₂ alkanes with a bifunctional catalyst comprising platinum (Pt) or palladium (Pd) and at least one other metal (M) to provide an alloy (Pt-M or Pd-M) containing at least 0.1 wt % of the platinum (Pt) or palladium (Pd), based on a total weight of the catalyst; a silica or alumina support; and an acidic zeolite, at a temperature of about 350° C. to 700° C. to provide a liquid product having a boiling point of 38° C. to 205° C.
2. The method according to paragraph 1, wherein the metal (M) is selected from the group consisting of Mn, Cr, V, Fe, Co. Ga, Sn, In, Bi, Zn and Sb.
3. The method according to paragraph 1 or 2, wherein the metal (M) is zinc (Zn).
4. The method according to any paragraph 1 to 3, wherein the acidic zeolite is ZSM-5.
5. The method according to any paragraph 1 to 4, wherein the one or more C₂ to C₁₂ alkanes consists essentially of C₂ to C₆ alkanes.
6. The method according to any paragraph 1 to 5, wherein the one or more C₂ to C₁₂ alkanes consists essentially of ethane and propane gas.
7. The method according to any paragraph 1 to 6, wherein the liquid product comprises one or more olefins, one or more paraffins and one or more aromatics.
8. The method according to any paragraph 1 to 7, wherein the reaction takes place in a single reactor using the bifunctional catalyst only.
9. The method according to any paragraph 1 to 8, wherein the liquid product has a boiling point of 50° C. to 200° C.
10. The method according to any paragraph 1 to 9, wherein the liquid product comprises less than 3 wt % ethane.
11. The method according to any paragraph 1 to 10, wherein the liquid product comprises about 5 wt % to about 80 wt % of one or more olefins, about 5 wt % to about 80 wt % of one or more paraffins; and about 10 wt % to about 90 wt % of one or more aromatics.
12. The method according to any paragraph 1 to 11, wherein the catalyst comprises about 0.1 wt % to about 10 wt % of the platinum (Pt) or palladium (Pd), based on a total weight of the catalyst.
13. The method according to any paragraph 1 to 12, wherein the catalyst comprises about 0.5 wt % to about 5 wt % of the platinum (Pt) or palladium (Pd), based on a total weight of the catalyst.
14. The method according to any paragraph 1 to 13, wherein the catalyst comprises about 0.05 wt % to about 15 wt % of the at least one other metal (M), based on a total weight of the catalyst.
15. The method according to any paragraph 1 to 14, wherein the catalyst comprises about 0.5 wt % to about 10 wt % of the at least one other metal (M), based on a total weight of the catalyst.
16. The method according to any paragraph 1 to 15, wherein a ratio of the total combined weight of the alloy and the support to the total weight of acidic zeolite is 0.02 to 10.
17. The method according to any paragraph 1 to 16, wherein a ratio of the total combined weight of the alloy and the support to the total weight of acidic zeolite is 0.1 to 2.

All patents and patent applications, test procedures (such as ASTM methods, UL methods, and the like), and other documents cited herein are fully incorporated by reference to the extent such disclosure is not inconsistent with this disclosure and for all jurisdictions in which such incorporation is permitted.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, meaning the values take into account experimental error, machine tolerances and other variations that would be expected by a person having ordinary skill in the art.

The foregoing has also outlined features of several embodiments so that those skilled in the art can better understand the present disclosure. Those skilled in the art should appreciate that they can readily use the present disclosure as a basis for designing or modifying other methods or devices for carrying out the same purposes and/or achieving the same advantages of the embodiments disclosed herein. Those skilled in the art should also realize that such equivalent constructions do not depart from the spirit and scope of the present disclosure, and that they can make various changes, substitutions, and alterations herein without departing from the spirit and scope of the present disclosure, and the scope thereof is determined by the claims that follow.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1. A method for converting lower alkanes to higher liquid products, comprising:
reacting one or more C₂ to C₁₂ alkanes with a bifunctional catalyst comprising platinum (Pt) or palladium (Pd) and at least one other metal (M) to provide an alloy (Pt-M or Pd-M) containing at least 0.1 wt % of the platinum (Pt) or palladium (Pd), based on a total weight of the catalyst; a silica or alumina support; and an acidic zeolite, at a temperature of about 350° C. to 700° C. to provide a liquid product having a boiling point of 38° C. to 205° C.

2. The method of claim 1, wherein the metal (M) is selected from the group consisting of Mn, Cr, V, Fe, Co, Ga, Sn, In, Bi, Zn and Sb.

3. The method of claim 1, wherein the metal (M) is zinc (Zn).

4. The method of claim 1, wherein the acidic zeolite is ZSM-5.

5. The method of claim 1, wherein the one or more C₂ to C₁₂ alkanes consists essentially of C₂ to C₆ alkanes.

6. The method of claim 1, wherein the one or more C₂ to C₁₂ alkanes consists essentially of ethane and propane gas.

7. The method of claim 1, wherein the liquid product comprises one or more olefins, one or more paraffins and one or more aromatics.

8. The method of claim 1, wherein the reaction takes place in a single reactor using the bifunctional catalyst only.

9. The method of claim 1, wherein the liquid product has a boiling point of 50° C. to 200° C.

10. The method of claim 1, wherein the liquid product comprises less than 3 wt % ethane.

11. The method of claim 1, wherein the liquid product comprises about 5 wt % to about 80 wt % of one or more olefins, about 5 wt % to about 80 wt % of one or more paraffins; and about 10 wt % to about 90 wt % of one or more aromatics.

12. The method of claim 1, wherein the catalyst comprises about 0.1 wt % to about 10 wt % of the platinum (Pt) or palladium (Pd), based on a total weight of the catalyst.

13. The method of claim 1, wherein the catalyst comprises about 0.5 wt % to about 5 wt % of the platinum (Pt) or palladium (Pd), based on a total weight of the catalyst.

14. The method of claim 1, wherein the catalyst comprises about 0.05 wt % to about 15 wt % of the at least one other metal (M), based on a total weight of the catalyst.

15. The method of claim 1, wherein the catalyst comprises about 0.5 wt % to about 10 wt % of the at least one other metal (M), based on a total weight of the catalyst.

16. The method of claim 1, wherein a ratio of the total combined weight of the alloy and the support to the total weight of acidic zeolite is 0.02 to 10.

17. The method of claim 1, wherein a ratio of the total combined weight of the alloy and the support to the total weight of acidic zeolite is 0.1 to 2.
